(19) United States
(12) Patent Application Publication
(10) Pub. No.: US 2005/0107726 A1
(43) Pub. Date: May 19, 2005

(54) Remote monitoring of an instrumented orthosis

(76) Inventors: Duane P. Oyen, Maple Grove, MN (US); Timothy N. Tracey, Wayzata, MN (US)

Correspondence Address: Patterson, Thuente, Skaar & Christensen, P.A., 4800 IDS Center, 80 South 8th Street, Minneapolis, MN, 55402-2100, US

(21) Appl. No.: 10/819,092

(22) Filed: Apr. 6, 2004

Related U.S. Application Data
(63) Continuation of application No. 09/382,433, filed on Aug. 25, 1999, now Pat. No. 6,872,187.

(51) Int. Cl. A61F 5/00

(52) U.S. Cl. 602/16

(57) ABSTRACT

An instrumented orthosis is interfaced to a telecommunication system such that a patient and health care professional can exchange communications including the monitoring of the instrumented orthosis. Thus, a health care can evaluate rehabilitation treatments based on an instrumented orthosis from a remote location while obtaining real-time feedback from the patient. The communications can include audio and/or video transmissions.

### CROSS REFERENCE TO RELATED APPLICATIONS

This application is a continuation of copending U.S. patent application Ser. No. 09/382,433 to Stark et al., entitled “Orthoses For Joint Rehabilitation,” incorporated herein by reference.

### BACKGROUND OF THE INVENTION

The invention relates to a system incorporating telecommunication capability to assist with rehabilitation involving an instrumented orthosis.

Both muscles and bones should be exercised to maintain strength. Also, bone fractures that are exposed to permissible weight bearing stress often heal more predictably and more rapidly than fractures that are not stressed at all. Improved healing based on application of appropriate stress is also believed to be true for connective tissue, such as ligaments and certain cartilage.

Suitable stress can be applied to the tissue by the performance of selected exercises. For example, isometric exercises generally involve the exertion of force against a relatively immovable object. To perform isometric exercises, a restraining device can be used that has a substantially unchanging position for the duration of a particular exercise routine. Isotonic exercises involve exertion against the same weight or resistance through a range of motion. Isokinetic exercise is designed to mimic exertions that take place on a playing field or the like. When performing isokinetic exercises in a simulated environment, a machine is used to provide resistance in direct proportion to the exertion of the exerciser.

Isometric exercises are particularly useful with painful injuries to lower the risk of further injury. If performed in a controlled manner, isometric exercises can be performed earlier in the recuperation period to speed recovery. As the patient's recovery progresses, isotonic exercises or other exercises can be used to reestablish a desired range of motion about a joint. As recovery progresses eventually the patient is able to perform a full range of exercises.

A difficulty with the application of stress to an injured joint is that the application of excessive stress can further injure the joint rather than assist with the healing. Thus, the exercises need to be carefully planned to provide appropriate amounts of stress. Also, the performance of the exercises should be monitored closely by a physician, physical therapist or other appropriate health care professional to reduce the risk of injury. The need to carefully plan and closely monitor the exercises provides a cost and motivation barrier to accessing desirable amounts of rehabilitation exercise.

Instrumented braces provide for the performance of exercises with immediate feedback. The feedback can prevent the patient from applying injurious forces and can prompt the patient to begin exercising. Instrumented braces can also have the capability of storing the patient's exercise performance parameters for later downloading to a health care professional for evaluation.

### SUMMARY OF THE INVENTION

In a first aspect, the invention pertains to an article comprising an instrumented orthosis and a telecommunication station, wherein the instrumented orthosis is connected to the telecommunication station for the transfer of information, and wherein the telecommunication station by way of a network transmits performance values from the instrumented orthosis while exchanging oral communications during a telecommunication session.

In another aspect, the invention pertains to a method of rehabilitating an injured joint, the method comprising exchanging transmissions over a telecommunication channel, the transmissions including real-time communications between a patient and a health care professional and values from an instrumented orthosis interfaced with a telecommunication station.

In a further aspect, the invention pertains to a method of adapting an instrumented orthosis for real time, remote evaluation by a health care professional, the method comprising interfacing the instrumented orthosis with a telecommunication station.

In addition, the invention pertains to a method of evaluating output from an instrumented orthosis, the method comprising producing a computer database including data from the performance of a plurality of patients using a comparable instrumented orthosis.

Furthermore, the invention pertains to a method of evaluating treatment with an instrumented orthosis, the method comprising exchanging communications between the patient and a health care professional by way of a video teleconference over a computer network.

### BRIEF DESCRIPTION OF THE DRAWINGS

FIG. 1 is a schematic view of a system involving an instrumented orthosis and a telecommunication device.

FIG. 2 is a schematic, perspective view of a simple instrumented orthosis.

FIG. 3 is a schematic, perspective view of an instrumented orthosis designed to fit around a patient's joint.

FIG. 4 is a schematic, top view of an orthosis controller.

FIG. 5 is a schematic, top view of a microprocessor based orthosis controller.

FIG. 6 is a schematic view of an auxiliary communications network.

FIG. 7 is a schematic view of a real-time, remote rehabilitation system based on an instrumented orthosis and a telecommunication network.

FIG. 8 is a flow chart outlining a preferred embodiment for the operation of a network based telecommunication device interfaced with a microprocessor based instrumented orthosis.

### DETAILED DESCRIPTION OF THE PREFERRED EMBODIMENTS

Instrumented orthoses can be interfaced with a telecommunication device to provide for real-time, remote monitoring of rehabilitative treatment by a health care professional, e.g., physician or physical therapist. In preferred embodiments, output from the orthosis is sent along the communication channels of the telecommunication device, although the orthosis output can be sent along an alternative communication pathway. A health care professional can receive the information from the orthosis and communicate with the patient to simulate the evaluation of the patient in the office of the health care professional.

Telecommunication as used herein includes teleconferencing relating to two or more remote locations transmitting audio communications and videophones/videoconferencing relating to two or more remote locations transmitting video and, generally, audio. Using the telecommunication approaches described herein, problems and concerns can be addressed quickly and efficiently without the expenditure of considerable effort and cost involved in scheduling a personal visit by the patient to the health care professional. Because of reduced costs and increased efficiencies, the patient can have increased numbers of sessions with the health care professional remotely. Also, family members of the patient can be involved in the remote sessions to assist patients who are limited in mobility.

An instrumented orthosis generally includes a support portion that is secured around a body portion of a patient. The orthosis can be instrumented with various transducers to provide output regarding patient manipulation of the orthosis. For example, the transducers can measure the strain within the orthosis, or the transducers can operate as position sensors to detect motion of the orthosis. Similarly, the orthosis can include energy propagating transducers that apply therapeutic transmission to the patient. In addition, the transducers can involve a motor for joint flexing, which can be part of a Continuous Passive Motion (CPM) device.

The instrumented orthosis includes a controller that generally provides a detectable output to the patient related to the value of the forces applied by the patient against the orthosis. The controller can further include an analog-to-digital (a/d) converter to provide a digital signal related to the analog transducer output and/or a digital-to-analog (d/a) converter to transform a digital signal into an analog signal to drive an energy propagating transducer. In preferred embodiments, digital signals can be directed to and/or from a microprocessor within the controller for storage, manipulation and/or transmission to a health care professional. Alternatively, a processor forming part of a telecommunication device can be used to perform desired functions with digital and/or analog signals relating to orthosis performance.

A telecommunication device involves data transmission of personal communication, generally in a digital format. Generally, the telecommunication transmissions are facilitated by computers, such as personal computers, at each end of the transmission. The communications can be transmitted by satellite relay, microwave relay, standard phone lines, digital phone lines (as part of an integrated services data network (ISDN) or a broadband T1 line or similar connection), other similar communication pathways or a combination thereof. The communications can include audio and/or video transmissions. In preferred embodiments, audio and video transmissions are included such that the patient and health care professional can communicate in a reasonable facsimile of a personal interaction from a remote location. In other embodiments, only a video transmission of the patient and audio transmissions by both the patient and health care professional are sent to the doctor along with the data transmission.

Preferred communication pathways include the internet, where a host web site functions as the telecommunication controller. The web site can provide a variety of other functions including facilitation of data transfer without patient or health care professional coordination. Also, the web site can provide for maintenance and access to a patient data base.

Communication signals and signals related to transducer response can be transmitted from the patient to a health care professional. Similarly, communications from the health care professional are transmitted back to the patient. Real-time communications take place between individuals such that they have a personal/live exchange while they are participating in a single session. In preferred embodiments, the instrumented orthosis can be interfaced directly with the telecommunication device such that the digitized output related to the transducer values can be sent along with the communication signals. Also, in preferred embodiments, the health care professional can send a digital transmission to reprogram a microprocessor based orthosis controller to instruct the patient to perform modified exercises with the orthosis.

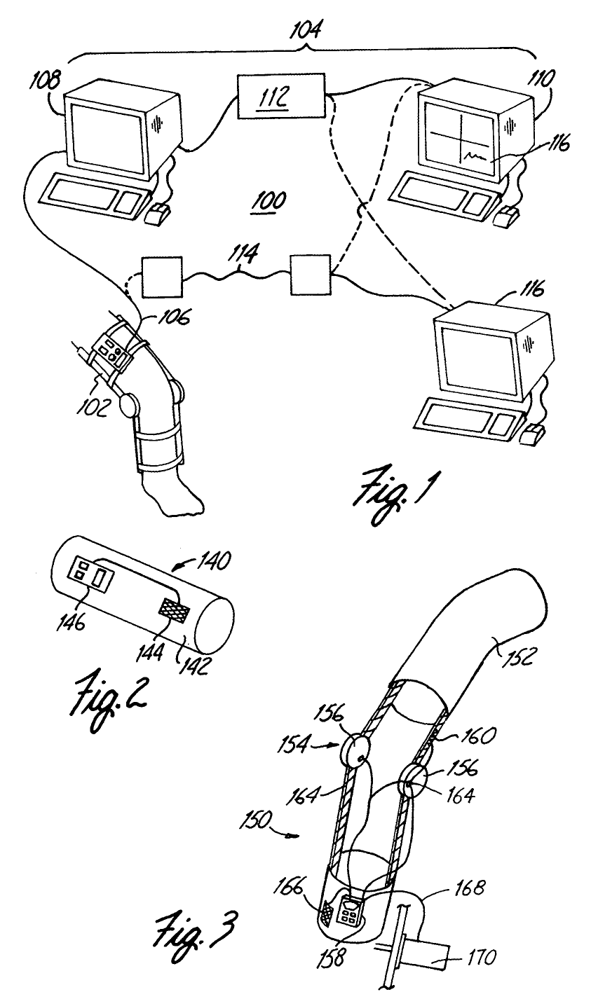

Referring to FIG. 1, a real-time remote monitoring system 100 includes an instrumented orthosis 102, a telecommunication/networking device 104 and an orthosis interface 106. Telecommunication device 104 includes a first computer 108, a second computer 110, a communication network 112, an optional second communication network 114 and a remote monitoring display 116. First computer 108 is located at the location of the patient, while second computer 110 is located at the location of the health care professional. First computer 108 and second computer 110 transfer data by way of communication network 112.

Orthosis interface 106 can connect to first computer 108 or to optional second communication network 114 (connection shown in phantom lines). Second communication network 114 provides an alternative pathway for the transfer of data from the instrumented orthosis at the patient's location to the remote location of a health care professional. Remote monitoring display 116 provides a graphic display of the data from the instrumented orthosis at the health care professional's remote location. Remote monitoring display 116 can be embodied in a variety of different formats such as a separate window on a display connected with second computer 110 or as a stand alone device, which can be connected to another computer. Alternative connections at the location of the health care professional are shown in phantom lines. Further details on such systems and components are described below.

Instrumented Orthoses and Orthoses Interfaces

An instrumented orthosis is suitable for the performance of monitored exercises by a patient and/or the propagation of therapeutic energy fields. Referring to FIG. 2, an instrumented orthosis 140 generally includes a support portion 142, a transducer 144 and a controller 146. Support portion 142 is designed to be secured on a selected body portion. A variety of constructions can be used for the support portions 142 such that a support portion properly supports the respective body portion. For example, a support portion can include one or more frame members, such as two frame members that extend on either side of the body portion. Straps, rigid connectors or the like can be used to connect the two frame member to hold the support portion in place around the body portion. The straps can be secured to the frame members with any of a variety of fasteners, such as snaps, buckles, clamps and hook and loop fasteners.

In alternative embodiments, a support portion surrounds the corresponding body portion. These support portions generally are rigid and can be constructed from a variety of materials. Preferred materials for the construction of rigid support portions include, for example, molded plastic shells, plaster, heat moldable thermoplastics, heat shrink plastic and other cast forming materials. Rigid support portions can be premolded in various sizes, such that a particular size is selected “off-the-self” based on measurements of the patient. Alternatively, rigid support portions can be constructed to custom fit a particular patient. Further description of the construction of support portions is found in copending and commonly assigned U.S. Provisional Application Ser. No. 60/098,779 to Stark et al., entitled “ORTHOSES FOR JOINT REHABILITATION,” incorporated herein by reference, hereinafter “application 60/098,779”. Application 60/098,779 is incorporated by reference in its entirety as well as, specifically, for the various features for which it is particularly referred to herein.

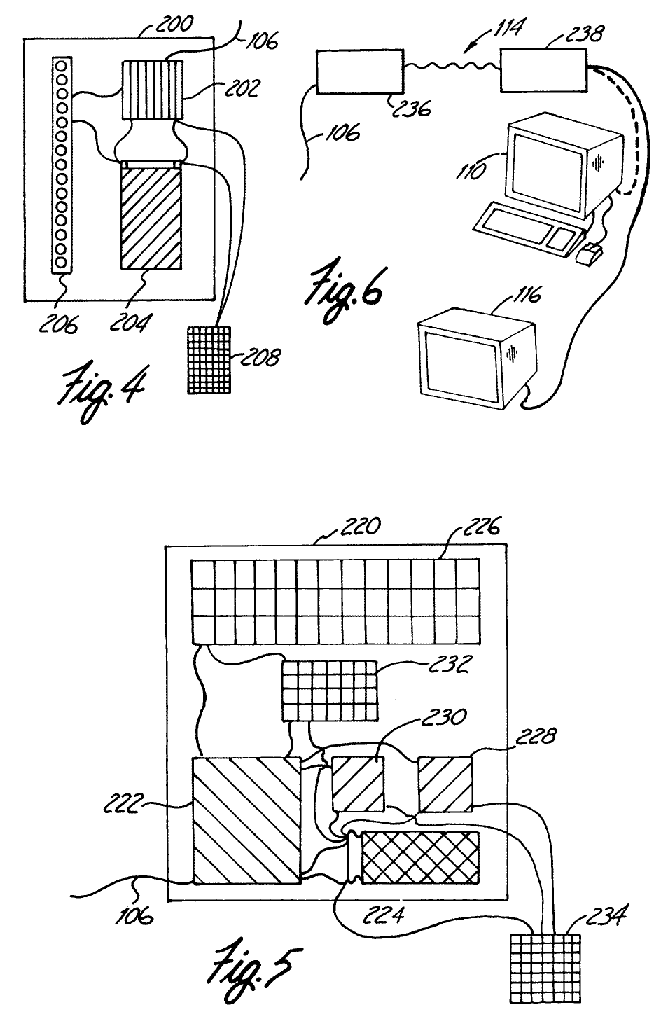

In preferred embodiments, instrumented orthosis 140 functions as an orthopedic restraining device that restrains flexibly connected body portions. Referring to FIG. 3, instrumented orthosis 150 includes a plurality of support portions 152 connected by a hinge 154. Suitable structures of hinge 154 may depend on the structure of the corresponding support structures. For example, a suitable hinge 154 can include two or more hinge units 156 connecting different frame members that rotate together around a joint, as shown in FIG. 3. In other constructions, hinge 154 has a single hinge unit that meets frame members at the joint.

Alternatively, support portions 152 can be connected with an articulating hinge. An articulating hinge can be made with resilient collapsible materials analogous to a bendable straw, sliding sections that can slide past each other to articulate, or other similar constructions. Sliding sections can be locked relative to one another by way of clamps attached to slots defining a range of motion, where the clamps are tightened manually with wing nuts or the like, or electronically with solenoids or the like.

Regardless of the structure of hinge 154, preferred hinges 154 are capable of locking and unlocking to provide for adjustment of the angle of the hinge and for support of the joint without the risk of undesired bending. Mechanical and electromechanical hinges capable of selectively locking and unlocking are described in U.S. Pat. No. 5,484,389 to Stark et al., incorporated herein by reference. In particularly preferred embodiments, hinge units 156 are capable also of adjustment to yield a selected resistance to rotation about the hinge for the performance of a variety of exercises. A suitable electromechanical hinge with variable resistance controllable by way of a control unit is described in published PCT application WO 96/36278, entitled “An Orthopedic Device Supporting Two or More Treatment Systems and Associated Methods,” incorporated herein by reference. A preferred embodiment of a mechanical hinge with variable resistance/friction is described in application 60/098,779.

Referring to FIG. 3, instrumented orthosis 150 generally includes one or a plurality of transducers connected to a controller 158. Suitable transducers include, for example, strain gauges 160, angle sensors 164, energy propagating transducers 166, or motorized flexing devices 168, such as a continuous passive motion apparatus. Strain gauges 160 can be calibrated to measures forces applied during isometric or isotonic exercises. Angle sensors 164 are used to measure rotation of a hinge/joint for the performance of any of a variety of exercises. Angle sensors 164 can be based on one or more different measurement approaches, such as angularly dependent variable resisters, digital optical transducers, hall effect magnetometers or simple switches with angle dependent contacts, to provide an angle dependent measurable parameter. Strain gauges 160 and angle sensors 164 are described further in application 60/098,779.

Energy propagating transducers can transmit ultrasound radiation and/or electromagnetic radiation for therapeutic purposes. It is known that propagating radiation of selected frequencies helps to speed healing of certain injuries. Energy propagating transducers used in instrumented orthoses and combinations of various transducers are described further in published PCT application WO 96/36278, entitled “An Orthopedic Device Supporting Two or More Treatment Systems and Associated Methods,” incorporated herein by reference.

Continuous passive motion (CPM) devices provide for range-of-motion exercises using a motor 170. Because of the use of motor 170, the exercises are passive in the sense that the patient's joint is moved without the need for applied forces by the patient. The orthosis controller can operate a CPM device or the like to flex a patient's joint and/or receive input from the CPM device regarding the parameters of the passive motion activity. The motor provides an effective transducer of electrical signals into physical motion of the patient's joint(s). By their nature passive motion devices involve a platform that interfaces with support portions for corresponding body portions near the joint. CPM devices are described further in, for example, U.S. Pat. No. 5,239,987 to Kaiser et al., entitled “ANATOMICALLY CORRECT CONTINUOUS PASSIVE MOTION DEVICE FOR A LIMB, incorporated herein by reference, and U.S. Pat. No. 4,934,694 to McIntosh, entitled “COMPUTER CONTROLLED EXERCISE SYSTEM,” incorporated herein by reference.

When forces are applied by the patient against the orthosis during exertion/exercise, the orthosis tends to change position relative to the patient's joint. This shifting reduces the effectiveness of exercises being performed with the orthosis and may necessitate realignment of the orthosis for proper fit. The orthosis can be designed to reduce or eliminate this shifting.

A first approach to prevent a knee orthosis from slipping during exercise is to construct the orthosis with indentations in the femur supracondylar area just above the knee. An alternative solution involves the use of additional securing cuffs. Securing cuffs are designed to be tightened more during exercise routines to help secure the orthosis relative to the joint. Securing cuffs include a gripping element and, for example, can be placed against the leg above the knee such that when tightened, the gripping element applies pressure above the kneecap and pushes on the knee without pushing on the vasculature and lymphatic drainage posteriorly. In other embodiments, the securing cuffs can be appropriately placed. Cuffs can be tightened with a variety of fasteners including hook and loop fasteners.

Another approach to securing the orthosis involves securing the orthosis to a belt by way of one or more straps. Still another approach involves reducing the friction of the surface contacting the orthosis or part of the orthosis, for example, using a high friction, polymer sleeve. Still another approach to securing the orthosis involves the placement of crossed straps behind the joint. The straps apply forces that tend to maintain the straps in the fold of the joint. Furthermore, for a knee orthosis, the orthosis can end with a heal cup with a strap or the like around the foot to hold the bottom of the orthosis around the heal of the foot and to fix the hinge at the knee.

With any of these approaches for inhibiting orthosis motion during use, the method preferably distributes the restraining forces sufficiently such that no portion of the skin is subject to excessive pressures that could bruise the skin as well as damage or interfere with neural or circulatory functions. Inhibition of orthosis slippage is described further in application 60/098,779.

Referring to FIG. 4, a controller 200 includes, for example, an a/d (d/a) converter 202, a power supply 204 and a display 206. A/D (d/a) converter 202 is connected to power supply 204, display 206 and transducer 208. If transducer 208 produces an analog signal, a/d converter 202 generally amplifies the signal and produces a corresponding digital signal. The digital signal can be output to orthosis interface 106 directly or following further manipulation within controller 200. If transducer 208 is an energy propagating transducer, a digital signal from orthosis interface 106 is converted into an analog signal that is directed to transducer 208 at the appropriate frequency.

Power supply 204 generally is a primary or secondary, i.e., rechargeable, battery. Alternatively, for telecommunication activities power supply 204 can be a line connected to the telecommunications device. If the telecommunications device includes a personal computer or the like, a 5 volt connection can be made by way of the keyboard port. Suitable plugs are available for tapping off of the keyboard port such that the keyboard is plugged into the connection for the peripheral device, in this case the controller 200. Display 206 can receive an analog or digital signal from a/d (d/a) converter 202. Display 206 can include a light display, a needle display, a digital display or the like. Display 206 includes a suitable driver that may or may not further amplify the signal.

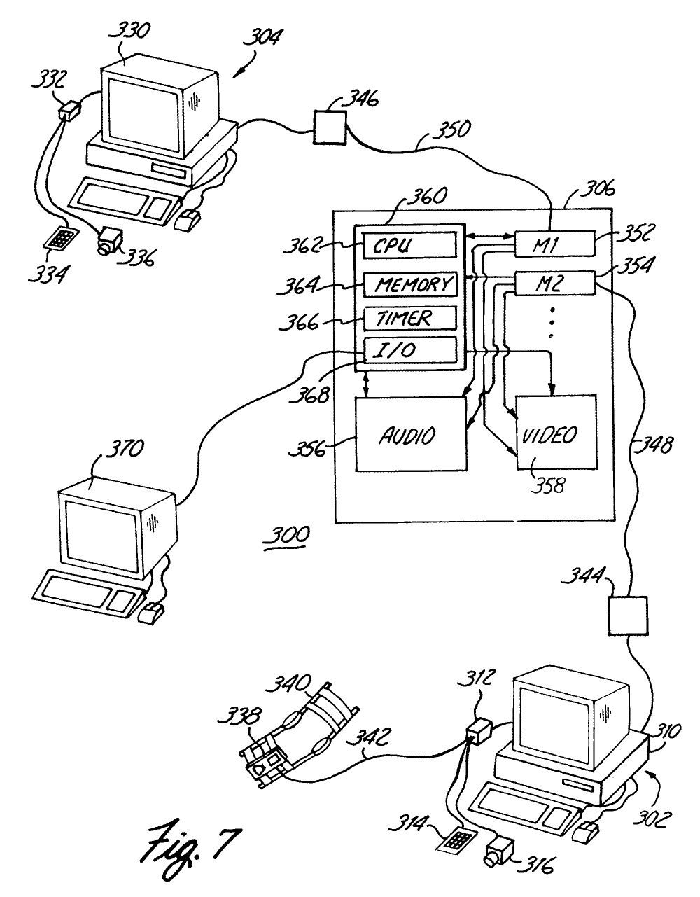

In a preferred embodiment, controller 220 includes a microprocessor 222, a power supply 224, a digital display 226, one or more a/d converters 228, one or more d/a converters 230 and an input 232, as shown in FIG. 5. A/D converter 228 and/or d/a converter are connected to transducer 234. Microprocessor 222 is connected to orthosis interface 106. Multichannel a/d or d/a converters can be used in place of multiple a/d or d/a converters. A preferred embodiment of an a/d converter 228 for amplifying and digitizing values from a strain gauge is described in application 60/098,779.

The digital processor can be programmed in any of a variety of computer languages including, for example, basic, assembler, C, C++ and the like. Microprocessor based controllers generally also include a real time clock, RAM and non-volatile storage such as SRAM or EEPROMO. Similarly, microprocessor based controllers generally include various input/output channels, such as, a keypad, a digital display, such as an LED display, data link to a transducer(s), and RS232 standard output for serial connection or modem access. The construction of microprocessor based controllers is described further in application 60/098,779.

In preferred embodiments, a microprocessor based controller prompts a patient for the performance of a treatment session. During the treatment session, the patient receives feedback regarding the performance of the treatment. Microprocessor based controllers can store and manipulate data regarding the treatment session. This information can be downloaded at suitable times to a computer of a health care professional, either remotely or by a direct connection.

As shown in FIG. 1, orthosis interface 106 can connect directly to a telecommunication device 104 or to a second communications network 114. Referring to FIG. 6, second communications network 114 includes a first signal transmitter/receiver 236 and a second signal transmitter/receiver 238. Signal transmitter/receivers 236, 238 can be modems, electromagnetic transmitter/receivers, or the like. The corresponding signals propagate through an appropriate medium, modem transmissions through phone lines or the like and electromagnetic (radio) transmissions through space. Second signal transmitter/receiver 238 is further connected to second microprocessor 110 or remote monitoring display 116.

Telecommunication

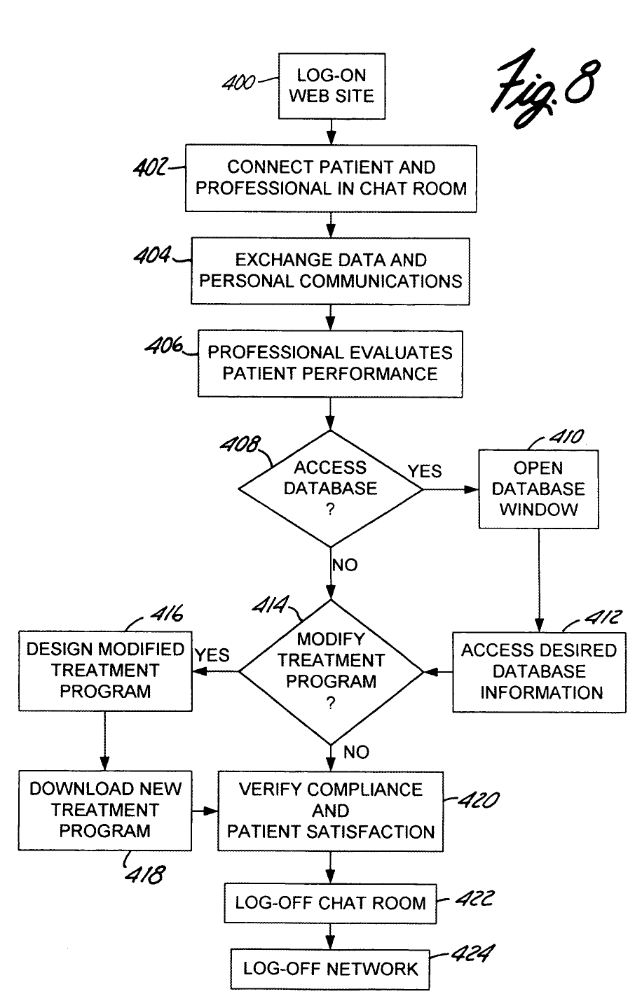

Referring to FIG. 7, an embodiment of a telecommunication/networking system 300 includes a patient telecommunication/networking station 302, a health professional telecommunication/networking station 304 and a telecommunication controller/host server 306. The patient telecommunication station 302 preferably includes a personal computer 310 or the like, a multiplexer 312, a microphone 314, and an optional video camera 316. Microphone 314 and video camera 320 are connected to multiplexer 322, which can be part of a board inserted within computer 310 or a stand alone device connected to computer 310. Multiplexer 322 can be removed and its functions carried out by software within computer 310 after input of digitized communications.

Health professional telecommunication station 304 includes a personal computer 330 or the like, a multiplexer 332, a microphone 334, and an optional video camera 336. Microphone 334 and video camera 336 are connected to multiplexer 332, which is connected to computer 330. The precise nature and order of connections within and from telecommunication stations 302, 304 can be altered for compatibility and for convenience.

Orthosis controller 338 of an instrumented orthosis 340 connect by way of serial connection 342 to multiplexer 312. As noted above, instrumented orthosis 340 can transmit relevant data by a separate communication pathway than the pathway used for the audio/video communication. FIG. 7 displays a preferred embodiment, where the data from instrumented orthosis 340 is transmitted on in series on the same communications pathway as the audio/video signal.

Computers 310, 330 interface with communication devices 344, 346, such as a modem or a high speed communication line. Ultimately, communication devices 342, 346 interact with network connections 348, 350, such as a satellite transmission network, a microwave communication network, the internet or other similar network. Network connections 348, 350 provide access to telecommunication controller 306. While computers 310, 330 can serve as telecommunication controller 306 with suitable modifications, telecommunication controller 306 preferably is a distinct unit.

Telecommunication controller 306 includes multiplexers 352, 354. Additional multiplexers can be used to provide for interfacing with additional telecommunication stations. Multiplexers 352, 354 feed into audio mixer 356 and video selector 358. Multiplexer 354 further feeds into processor 360 to provide data from the instrumented orthosis 316 to processor 360. Audio mixer 356 and video selector 358 interface with processor 360 to provide for control of the exchange of audio and video data between work stations 302, 304. Processor 360 includes a central processing unit 362, volatile and/or nonvolatile memory 364, a timer 366 and input/output ports 368. Processor 360 can use a Windows™, Macintosh™, Unix™, or other operating system. Processor 360 connects to a display 370, generally through a serial connection.

The coordination of the audio and video portions of the communications by a telecommunication controller is described further in U.S. Pat. No. 5,801,756 to Iizawa, entitled “Multipoint Video Conference System,” incorporated herein by reference, and in U.S. Pat. No. 5,751,959 to Sato et al., entitled “Communication Terminal Supervisory System and Communication Method,” incorporated herein by reference.

Generally, raw data or data following some analysis can be downloaded by controller 338. Orthosis controller 338 can perform some initial data analysis to reduce the amount of data that must be stored and transferred. Similarly, data transferred by orthosis controller 338 can be analyzed by computer 310 prior to transmission to transmission to telecommunications controller 306. If computer 310 is used to analyze the data prior to transmission, multiplexer 322 should be connected appropriately. In any case, the amount of data is generally small relative to the amount of data associated with standard audio/video signals. Packets of data can be transmitted/written by orthosis controller 338 in format for an RS232 port or other type of serial port or parallel port using suitable formats including standard formats. Possible display formats for data from an instrumented orthosis is described in application 60/098,779.

The orthosis controller 338 can be designed to assist with the telecommunication process. In particular, the microphone and/or video camera can be connected directly to controller 338, such that controller 338 performs the necessary multiplexing. A specific preprocessor can be used as a data manager. The controller can be connected to a personal computer or the like by way of an RS-232 connection or other suitable protocol through a serial connection or a parallel connection with power directed from the pc to the controller for telecommunication applications.

In some preferred embodiments, networking by way of network connections 344, 346 takes place by way of the internet or other comparable system that may supersede the internet. A rapidly growing number of individuals have access to the internet from their homes. The internet provides extremely convenient and inexpensive access to telecommunication capability that is available to a growing number of people from their homes. For valuable communication between a patient and a health care professional, suitable telecommunication needs just moderate quality communication available now over the internet.

The telecommunication equipment can be rented to a patient with internet access for use during a rehabilitation period, if the patent does not already own the necessary equipment. In preferred embodiments, the patient can be supplied with a laptop computer set-up for telecommunication, such as internet access, a microphone/camera and software for linking to the orthosis, such that difficulties with set-up are reduced or eliminated. Thus, access can be provided inexpensively and conveniently to all patients. Then, all patients with a phone line can take advantage of these telecommunication capabilities described herein.

In one preferred approach, a provider hosts a web site that can be accessed by both the patient and the health care professional. World wide web servers are routinely set up to operate chat rooms between individuals. After proper identification of the patient and health care professional by user names and/or passwords, they are linked in a “chat room” represented by a window on their respective display screens. Internet service provider software and web browsers generally enable host web servers to set-up chat room windows on the remote user. Inexpensive hardware/software packages are commercially available to adapt personal computers running windows to provide telecommunication capabilities to the health care professional and the patient.

While the discussion has focused on the transmission of data from orthosis controller 338 to a remote location for analysis, in some preferred embodiments orthosis controller 338 can be reprogrammed by another computer using a serial connection. Using the telecommunication system 300, orthosis controller 338 can be programmed remotely by a health care professional during a telecommunication session. The reprogramming can be based on instructions provided by computer 330 of telecommunication station 304 or under input from computer 330 based on instructions transmitted by telecommunication controller/host server 306.

Remote Monitoring of Orthosis via Telecommunication

Networking capability by way of a telecommunication controller/host server 306 can be used also to facilitate transfer of data, maintenance of a patient database, access to databases and software, as well as telecommunication. A variety of approaches have been described for transferring data between an orthosis controller and a health care professional's computer. These approaches include, for example, modem-to-modem transfer, radio communication and direct transfer by way of an RS232 port, as described in application 60/098,779.

Telecommunication controller/host server 306 provides a particularly convenient alternative. With the configuration shown in FIG. 7 or a comparable arrangement, the patient can download the data to the controller 306. The data is stored on controller 306 for retrieval by the health care professional at their convenience. The data transfer can take the form of an e-mail attachment or the like. This eliminates the need for any direct coordination between the patient and the health professional. More generally, controller/server 306 can serve as a drop off point for communications of all types between patient and health care professional. If desired, these communications can take the form of e-mail or a bulletin board that is accessible with a password.

In preferred embodiments, data stored on controller/server 306 is separated from any patient identification and added to a patient database. A patient database provides a basis for improving therapy approaches by statistically evaluating treatment approaches and by permitting a comparison with results of other similar cases. In other words, the database can include real-time access to compilations of raw data, statistically analyzed data and/or comparative analyses. Preferably, the database is accessible at any time by the health care professional including during a telecommunication session with the patient.

Controller/server 306 can also provide access to software for either the telecommunication stations and/or an orthosis controller. For example, software upgrades based either on revisions of the software or modification indicated by treatment results can be directly downloaded over the network to the telecommunication stations. Similarly, software for orthosis controller 338 can be downloaded directly from controller/server 306 under the direction of the health care professional. The downloading of software to orthosis controller 338 can be performed as part of a telecommunication session or during a separate visit by the patient to the controller/server based on previous instructions from the health care professional.

The capability of having real-time, remote monitoring of the treatment program by way of the telecommunication capability potentially provides a variety of useful purposes. While a health professional generally shows a patient how to use the orthosis during a fitting of the orthosis, telecommunication capability enables the health professional to walk through the procedure again from a remote location to reassure the patient. This reassurance can improve patient compliance while reducing patient stress. Also, patient questions can be answered at an early time in the treatment program, such that the treatments are properly performed, without burdening the health professional.

In addition, if a patient is having difficulty with treatments, a telecommunication session provides for very efficient remote evaluation of the problem at a without requiring the patient to travel to the professional's office. Thus, scheduling difficulties can be reduced significantly. This provides a convenience to both the patient and to the health professional. While transducer data can be sent at roughly the same time as communications between the patient and the health care professional, the data from the orthosis can be sent prior to the start of a telecommunications session between the patient and the health care professional, if desired. Thus, the health care professional can review the orthosis data prior to the telecommunication session, preferably a teleconferencing session at an internet web page or the like.

A typical telecommunication session is outlined with a flow diagram in FIG. 8. First, the patient and health professional log-on 400 the web site. Based on user names, passwords or other information entered, the patient and health professional are connected 402 in a chat room. Through the chat room window, the patient and health professional exchange 404 personal communications and data regarding the performance of the treatment program. Generally, the health care professional opens another window to display the data from the instrumented orthosis, although other approaches are discussed above. Based on the personal communications and an examination of the orthosis data, the health professional evaluates 406 the patient performance.

To assist with the professional's evaluation, the health care professional can select 408 to access a patient database. If the professional decides to access the database, they open 410 another window for database access. The desired information is accessed 412 from the database. Whether the database is accessed or not, the health professional decides 414 whether or not the treatment program should be modified.

If the health professional decides to modify the treatment program, the modified treatment program is designed 416. Then, the modified treatment program is downloaded 418 to the orthosis controller 338. In preferred embodiments, orthosis controller 338 is responsive to remote programming. Thus, the reprogramming can be performed during the telecommunication session. If desired, the reprogrammed treatment program itself can be evaluated during the telecommunication session shortly after the reprogramming is performed.

Once any modifications are made to the programming of orthosis controller 338, the health professional verifies 420 the patient's expectations regarding compliance and their satisfaction 420. Upon completion of the telecommunication, the patent and health professional log-off 422 of the chat room and log-off 424 of the network.

The embodiments described above are intended to be illustrative and not limiting. Additional embodiments are within the claims below. Although the present invention has been described with reference to preferred embodiments, workers skilled in the art will recognize that changes may be made in form and detail without departing from the spirit and scope of the invention.

1. An article comprising an instrumented orthosis, a microphone and a telecommunication station comprising a plurality of input channels and an output channel, wherein the instrumented orthosis comprises a transducer that responds to forces or propagates forces within the orthosis, wherein the instrumented orthosis and the microphone are connected to the input channels of the telecommunication station, and wherein the telecommunication station can transmit from the output channel a multiplexed signal in digital format performance values from the instrumented orthosis over a network while exchanging oral communications with a person on the network during a telecommunication session, the telecommunication station exchanging performance values from the instrumented orthosis and oral communications with a telecommunications controller.

2. The article of claim 1 wherein the instrumented orthosis comprises a microprocessor based controller.

3. The article of claim 1 wherein the instrumented orthosis comprises energy propagating transducers.

4. The article of claim 1 wherein the telecommunication station comprises a multiplexer connected to the microphone, wherein the multiplexer is connected to the input channels and the multiplexed out is connected to the output channel.

5. The article of claim 1 wherein the telecommunication station comprises a video camera.

6. The article of claim 1 wherein the telecommunication station comprises a modem, which is connectable to a phone line.

7. The article of claim 6 wherein the telecommunication station can access a network server that can address a web site chat room enabling the simultaneous communication with a remote site and transfer of data from the instrumented orthosis.

8. The article of claim 1 wherein the telecommunications station comprises a microprocessor.

9. The article of claim 8 wherein the microprocessor performs multiplexing of the data from the input channels connect to the instrumented orthosis and oral communication from the microphone and the multiplexed output from the microprocessor is connected to the output channel.

10. A method of rehabilitating an injured joint, the method comprising exchanging transmissions in digital format over a telecommunication channel, the transmissions including interspersed real-time oral communications between a patient and a health care professional and values from an instrumented orthosis interfaced with a telecommunication station, the instrumented orthosis comprising a transducer that responds to forces within the orthosis wherein the values from the instrumented orthosis relate to the output of the transducer and the telecommunication station exchanging values from the instrumented orthosis and oral communications over the telecommunications channel with a telecommunications controller.

11. The method of claim 10 wherein the real-time communications comprise oral statements made by the patient and by the health care professional.

12. The method of claim 11 wherein the real-time communications further comprise video transmissions of the patient.

13. The method of claim 10 wherein the telecommunication channel comprises an internet connection.

14. The method of claim 10 wherein the telecommunication channel comprises a satellite.

15. The method of claim 10 wherein the telecommunication channel comprises a server that links the patient and health care professional.

16. The method of claim 10 wherein the instrumented orthosis comprises a microprocessor based controller.

17. The method of claim 16 further comprising remotely reprogramming the controller.

18. The method of claim 10 wherein the telecommunication station comprises a multiplexer connected to a microphone.

19. The method of claim 10 wherein the telecommunications station comprises a microprocessor that performs multiplexing of data from the instrumented orthosis and oral communication from a microphone.

20. The method of claim 10 wherein the telecommunication channel comprises a modem or a high speed communications line.
